(19) United States
(12) Patent Application Publication
(10) Pub. No.: US 2022/0313875 A1
(43) Pub. Date: Oct. 6, 2022

(54) PLATELET RELEASE SYSTEM AND PLATELET RELEASE METHOD

(71) Applicants: ETABLISSEMENT FRANCAIS DU SANG, LA PLAINE SAINT DENIS (FR); AVIGNON UNIVERSITE, AVIGNON CEDEX 1 (FR); INSTITUT NATIONAL DE LA SANTE ET DE LA RECHERCHE MEDICALE, PARIS (FR); UNIVERSITE DE STRASBOURG, STRASBOURG Cedex (FR)

(72) Inventors: Valentin DO SACRAMENTO, URMATT (FR); Yannick KNAPP, MARSEILLE (FR); Lea MALLO, STRASBOURG (FR); Catherine STRASSEL, STRASBOURG (FR)

(21) Appl. No.: 17/764,686

(22) PCT Filed: Oct. 12, 2020
(86) PCT No.: PCT/EP2020/078639

§ 371 Date: Mar. 29, 2022

(30) Foreign Application Priority Data
Oct. 11, 2019 (FR) FR1911303

(51) Int. Cl. A61M 1/02; C12N 5/00

(52) U.S. Cl. CPC A61M 1/029; C12N 5/0087; A61M 2202/0427

(57) ABSTRACT

The invention relates to a system for platelet release from a fluid comprising in particular megakaryocytic cells comprising cytoplasmic extensions.

The system allows to reproduce the pipetting process performed manually by means of a pipette, thereby permitting a continuous and automatic release of platelets.

This is performed on a large scale since the geometry of the system according to the invention allows to treat large volumes of fluid with a high flow rate (of the order of a plurality of liters per hour), thereby making it particularly suitable for use on an industrial scale.

The invention also relates to a method for platelet release from a fluid comprising in particular megakaryocytic cells comprising cytoplasmic extensions, said method being implemented by means of the abovementioned system.

### TECHNICAL FIELD OF THE INVENTION

The invention relates to a system for the platelet release from a fluid comprising in particular megakaryocyte progenitors, megakaryocytic cells comprising cytoplasmic extensions. The invention also relates to an assembly provided with a plurality of systems such as this. Finally, the invention relates to a method for continuous release of platelets by means of such a system. The invention is more particularly adapted to the in vitro production of blood platelets on an industrial scale.

### BACKGROUND

The in vitro production of blood platelets is meeting increasing needs in various medical applications. Currently, there are essentially two categories of systems intended to this specific purpose: the microfluidic systems and the “reservoir” systems. These categories of systems intended to release the platelets are derived from observations made in situ that show the need for a flow.

An example of a reservoir system is described in the document WO201909364 A1. The system is provided with means for stirring the fluid to be treated. It comprises a reservoir for the fluid comprising in particular the megakaryocytic cells and at least one means for stirring the fluid, the stirring causing the release of the platelets. This stirring is generated inside the reservoir itself, for example, by means of a vertical reciprocating paddle. The main disadvantage of such a system is that it is not adapted to operate under conditions which guarantee the quality of the platelets thus obtained, since it is not isolated from the external environment and the sources of contamination present in that environment, which makes it unsuitable for a medical use.

The microfluidic systems, as the name implies, are generally systems of micrometric dimensions (a few tens of microns to a few hundreds of microns at most) comprising a network of reservoirs and channels interconnected in a particular arrangement to perform different functions. Classically, they comprise a site dedicated to the culture of megakaryocytic cells or megakaryocytes, and optionally to the release of the platelets, the site being connected to a network of channels configured to extract/retrieve the platelets. These systems, most of which are biomimetic, seek to mimic the physiological environment to increase the platelet production.

Due to their small size, these microfluidic systems have two intrinsic limitations. The first is the low platelet flow rate that can be achieved by such systems, typically a few hundred microliters per hour (μL/h). As an example, a system with a flow rate of 200 μL/h would require 50,000 hours, or 5.7 years, to treat 10 L of fluid. It is then necessary to connect several systems in parallel to obtain a comparable total flow rate. However, in the above example the number of systems required would be very high, i.e. 50,000, and the complexity of the system would be further increased. At best such systems are only suitable for limited volumes of fluid samples.

In addition, the microfluidic devices described in the literature all implement a mechanism/method that “fix” the megakaryocytes and then extracts the platelets. This fixing phase is often based on the use of a drug or a chemical coating compound, which is a disadvantage.

More recently, a new method for releasing the platelets from megakaryocytic cells using pipetting has been developed. Such a method is described in the document Strassel et al. “Aryl hydrocarbon receptor-dependent enrichment of a high potential to produce propalets,” Blood, May 5, 2016, vol. 127, n° 18. In such a method, the process for releasing the platelets is a direct result of the pipetting. The pipetting is similar to a conventional pipetting in that a sample of a medium comprising megakaryocytic cells is taken by means of a pipette, except that the pipetting action is repeated as many times as necessary to create the stirring required to the release of the platelets. As a result of this method, platelets were detected in the treated fluid, validating the ability of pipetting to the release of the platelets, and with great ease of implementation. However, this method is not suitable for large-scale operation since the pipetting is by nature manual and therefore it is only suitable for treating limited volumes of fluid.

### SUMMARY OF THE INVENTION

The invention allows to overcome the aforementioned disadvantages and to this end proposes a system for releasing the platelets from a fluid comprising, in particular, megakaryocytic cells comprising cytoplasmic extensions, said system comprising:
a device comprising:
   a platelet release reservoir comprising a first opening and a second opening,
   a first fluidic connecting element attached at the level of said first opening and adapted to inject said fluid into said reservoir, said first connecting element comprising an orifice for injecting the fluid into the platelet release reservoir and a portion narrowing towards said orifice, there being an abrupt widening of cross-section between the injection orifice and the reservoir,
   a second fluidic connecting element attached at the level of said second opening, said second connecting element comprising an orifice for discharging the fluid,
a device for pumping the fluid in fluidic communication with the reservoir by means of the second fluidic connecting element or the first fluidic connecting element,
a power supply module for the pumping device,
a programming system configured to control the power supply module for the pumping device to implement one or more platelet release sequences designed to generate a continuous flow of the fluid between said injection orifice and said discharge orifice and vortex disturbances within the reservoir causing the fragmentation of the cytoplasmic extensions of the megakaryocytic cells.

The system according to the invention is thus configured in such a way as to reproduce the pipetting process performed manually by means of a pipette, thus allowing a continuous and automatic release of the platelets.

A further noteworthy point is the fact that the invention is implemented on a large scale because the geometry of the system according to the invention allows large volumes of fluid to be treated at a high flow rate (of the order of several liters per hour), which makes it particularly suitable for use on an industrial scale.

In addition, the system allows to achieve a number of platelets released from the megakaryocytic cells at least equivalent to that obtained from the manual pipetting, making it a system with a particularly high platelet release efficiency. This is achieved mainly due to the synergy between the shape of the elements of the system, i.e. in particular the conicity of the first fluidic connecting element and the large dimensions of the reservoir, and the flow velocity generated by the pumping device. This allows for the generation of vortex disturbances that are significantly of the size of the megakaryocytes within the reservoir.

According to various characteristics of the invention which may be taken together or separately:
the narrowing portion is conical;
the first connecting element comprises a longitudinal axis and the second connecting element comprises a longitudinal axis, said longitudinal axes being either intersecting or parallel and then separated by a non-zero distance, d;
the injection orifice has an opening diameter of less than 1 mm;
a ratio of the opening diameter of the injection orifice by a sectional width of the reservoir is between 0.02 and 0.1;
the ratio of the opening diameter of the injection orifice to the cross-sectional width of the reservoir is 0.05;
the discharge orifice has an opening diameter of less than 1 mm, a ratio of the opening diameter of the discharge orifice to a sectional width of the reservoir is between 0.02 and 0.1;
the ratio of the opening diameter of the discharge orifice to the cross-sectional width of the reservoir is 0.05;
the reservoir has a spherical shape;
the system comprises a source reservoir for the storage of the fluid, connected to the first fluidic connecting element for supplying the reservoir for platelet release;
the system comprises a reservoir for receiving the fluid connected to the second fluidic connecting element to collect said fluid intended to be sucked from the reservoir for platelet release;
the pumping device is located in the receiving reservoir;
said second connecting element further comprises a portion flared from said orifice for discharging the fluid towards the pumping device;
the system comprises one other device, said other device comprises:
   a platelet release reservoir comprising a first opening and a second opening,
   a first fluidic connecting element attached at the level of said first opening and adapted to inject said fluid inside said reservoir, said first connecting element comprising an orifice for injecting the fluid and a first narrowing portion so as to be able to accelerate the fluid, said first narrowing portion opening onto said injection orifice,
   a second fluidic connecting element attached at the level of said second opening, said second connecting element comprising an orifice for discharging the fluid,
said other device being arranged parallel to the first device and connected to the pumping device by means of the second of said other device;
the system comprises another device, the other device comprises:
   a platelet release reservoir comprising a first opening and a second opening,
   a first fluidic connecting element attached at the level of said first opening and adapted to inject said fluid inside said reservoir, said first connecting element comprising an orifice for injecting the fluid and a first narrowing portion so as to be able to accelerate the fluid, said first narrowing portion opening onto said injection orifice,
   a second fluidic connecting element attached at the level of said second opening, said second connecting element comprising an orifice for discharging the fluid,
said other device being arranged in series with the first device, said second connecting element of said other device being in fluidic communication with said first fluidic connecting element of said first device.

The invention further relates to a method for releasing platelets from a fluid comprising in particular megakaryocytic cells comprising cytoplasmic extensions, said method comprising the following steps, implemented by means of a system as previously described:

(100) providing a fluid comprising megakaryocytic cells suspended in said fluid, said megakaryocytic cells comprising cytoplasmic extensions,
(200) electrically powering the pumping device,
(300) controlling the programming system to initiate one or more platelet release sequences, the or each platelet release sequence being carried out so as to generate a continuous flow of the fluid between said injection orifice and said discharge orifice and vortex disturbances within the reservoir causing the fragmentation of the cytoplasmic extensions of the megakaryocytic cells.

Advantageously, during the step (200), a relative vacuum of between −10 kPa and −50 kPa is generated in the device.

### BRIEF DESCRIPTION OF FIGURES

Further objects, characteristics and advantages of the invention will become clearer in the following description, made with reference to the attached figures, in which:

FIG. 1a is a schematic representation of a system according to a first embodiment of the invention, a reservoir for the fluid being illustrated in section according to a side view;

FIG. 1b FIG. 1b is a schematic representation of an alternative embodiment of the system of FIG. 1a;

FIG. 1c is a close-up view of a reservoir equipping the system shown in FIG. 1b;

FIG. 2a FIG. 2a is a schematic representation of a system according to a second embodiment of the invention comprising a plurality of devices connected in series;

FIG. 2b FIG. 2b shows an exploded view of a system according to the invention comprising five devices connected in series;

FIG. 2c FIG. 2c shows an assembly as shown in FIGS. 2a and 2b, the assembly is here connected to a source reservoir and a reservoir for receiving the fluid;

FIG. 2d FIG. 2d is a schematic representation of an assembly according to a third embodiment of the invention, comprising a plurality of devices connected in parallel;

FIG. 3a FIG. 3a is a schematic representation of a method according to the invention:

FIG. 3b FIG. 3b is a schematic representation of the method of FIG. 3a in which the sub-steps for carrying out the method are illustrated;

FIG. 4a FIG. 4a illustrates a proplatelet megakaryocytic cell;

FIG. 4b FIG. 4b illustrates platelets obtained after the platelet release method according to the invention;

FIG. 5a is a comparative figure illustrating the platelet release efficiency obtained using a pipette and using a system according to the invention as a function of the relative vacuum within said system;

FIG. 5b FIG. 5b is an analysis of the functionality of the platelets obtained at a pressure of −30 kPa for native platelets (hollow bars) and cultured platelets (solid bars).

### DETAILED DESCRIPTION OF THE INVENTION

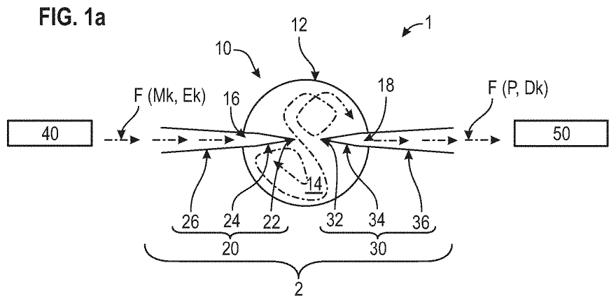

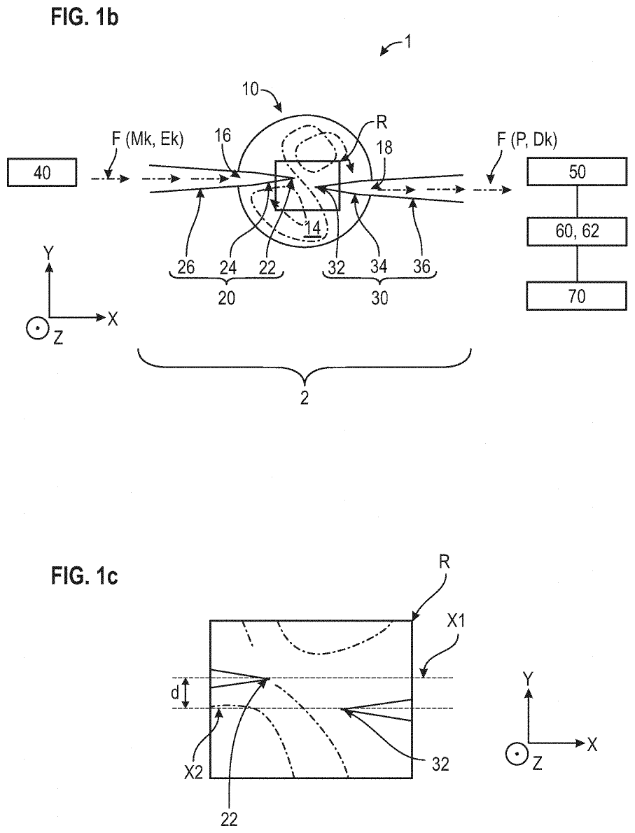

With reference to FIGS. 1a to 1c, the invention relates to a system 1 for the release of platelets P from a fluid F comprising megakaryocytic cells Mk.

The fluid F in question is, for example, a culture medium containing a population of cells obtained from immortalized or non-immortalized strains cells at different stages of differentiation, including megakaryocyte progenitors, megakaryocytes. The megakaryocytes or megakaryocytic cells are large blood cells (up to 100 μm and 30 μm in culture) which, when mature, have long extensions called cytoplasmic extensions or proplatelets.

The mechanisms involved in the formation of the blood platelets are still the subject of much research. Among them, the platelet release occurs during a process of fragmentation of the Mk megakaryocytes and/or cytoplasmic extensions, Ck, into platelets. This is a highly coordinated in vivo process that occurs naturally in the blood due to the force of the blood flow. The megakaryocytes Mk play an essential role as they are precursor cells. However, the process of release of the platelets in the body remains poorly described and there are still many questions about transendothelial passage and the precise role of the blood flow in the formation of the platelets. This process was reproduced in vitro by means of microfluidic systems, which allowed some in vivo mechanisms to be corroborated by microfluidic experiments. This process can also be reproduced in vitro by means of a pipette manually or by means of devices such as those presented in the introductory part of this description, which also allows a better understanding of the mechanisms involved in the release of the platelets (Strassel et al. 2016). The production of platelets in vitro provides a better understanding of the mechanisms involved in the formation of the platelets. However, in general, the efficiencies of the release of the platelets are lower than those obtained in vivo. The device proposed by the invention allows to mimic the platelet release process by manual pipetting disclosed by Strassel et al., without reproducing the reciprocate movement generated during the pipetting which is replaced by a continuous movement using the system 1. The invention thus aims to reproduce the fragmentation process of the megakaryocytes Mk and/or cytoplasmic extensions Ck on an industrial scale and thus allows to treat large volumes of fluid F (several liters per hour).

The system 1 according to the invention comprises a device 2, a pumping device 60 and a programming system 70 which will be described in the following sections.

The device 2 comprises a reservoir 10 of platelet release, a first fluidic connecting element 20 and a second fluidic connecting element 30.

The reservoir 10 of platelet release (hereinafter referred to as “reservoir 10”) has millimeter to centimeter longitudinal dimensions, i.e., longitudinal dimensions between 1 mm and a few centimeters. For comparison, the smallest dimension of the reservoir 10 of the system 1 according to the invention is the largest dimension of a conventional microfluidic system. This makes it a larger reservoir and larger capacity than known microfluidic systems.

In the example embodiment shown in FIG. 1a, the reservoir 10 is spherical in shape. It comprises a spherical wall 12 delimiting a cavity 14 within which the fluid F can circulate. In addition to being a zone for the circulation of the fluid F, the cavity is an area allowing a turbulent flow of the fluid F, i.e. allowing the formation of vortex disturbances of sizes at least equal to those of the megakaryocytes Mk. In other words, the cavity 14 by its dimensions, as previously described, on the one hand, and by its internal volume on the other hand, is configured so as to allow the formation of vortex disturbances within it. However, the reservoir 10 could be of any other shape, for example, parallelepipedal, cylindrical, etc., the most important being that said reservoir 10 comprises a cavity 14 of sufficient dimensions to allow the formation of the vortex disturbances. For example, for a fluid F comprising megakaryocytes Mk, the size of which, as we have seen, can reach 30 μm in culture, the cavity 14 therefore has much larger dimensions (millimeter to centimeter) since the fluid F comprises a plurality of megakaryocytic cells Mk, for example hundreds or even thousands.

The wall 12 comprises at least a first opening 16 and a second opening 18. The first opening 16 is dedicated to the fluidic connection with the first connecting element 20, while the second opening 18 is dedicated to the fluidic connection with the second connecting element 30. Preferably, they form the only openings in the wall 12, since the system 1 is closed.

The first fluidic connecting element 20 is a means for supplying the fluid F to the reservoir 10. In other words, it is an inlet for the fluid F within the reservoir 10. It is attached at the level of the first opening 16 of the reservoir 10. It comprises a connecting portion 26, a narrowing portion 24 and an orifice 22 of injection of the fluid in that order in the direction of flow direction of the fluid. The narrowing portion 24 thus opens onto the injection orifice 22. This being said, it should be noted that the connecting portion 26 is not indispensable as will be better understood in the following. Thus, in the example embodiment shown in FIG. 1a, the first fluidic connecting element 20 is attached at the level of the first opening 16 by means of the connecting portion 26, but may be attached to the reservoir 10 in other configurations. Indeed, it can just as easily be attached by means of the narrowing portion 24 as by means of the injection orifice 22. In the latter configuration, it will be understood that the injection orifice 22, being located at the level of the wall 12, may correspond to, i.e. coinciding with, the first opening 16 of the reservoir.

As mentioned earlier, the portion 24 is narrowing. The portion 24 is narrowing in that it narrows from the connecting portion 26 or, alternatively in the absence of a connecting portion 26, an end of the first connecting element 20 towards the orifice 22 of injection of the fluid. In other words, it narrows in the direction of the flow relative to a longitudinal axis X1 of said first connecting element 20 passing through the center of said orifice 22. The narrowing shape of said portion 24 allows the fluid F to be accelerated by the venturi effect. Incidentally, the fluid F entering the reservoir 10 therefore has a higher velocity compared to a fluid that would enter the system 1 through a portion 24 that does not have a flaring.

Advantageously, the narrowing of the narrowing portion 24 from the connecting portion 26 or, alternatively in the absence of a connecting portion 26, the end of the first connecting element 20 towards the orifice 22 of injection of the fluid may be substantially constant. The constancy of the narrowing allows to reduce the friction and further increase the acceleration of the fluid. In this regard, the narrowing portion 24 may be conical. Advantageously, the use of a conical portion 24 facilitates the connection with tubular, i.e. cylindrical, fluidic connecting means frequently sold commercially. By way of example, the portion 24 may have a pyramidal shape, a tetrahedral shape, etc. Most importantly, the narrowing portion 24 narrows from the connecting portion 26 of the first connecting element 20 toward the orifice 22 of injection of the fluid. This being the case, once discharged into the reservoir 10, the fluid F slows down substantially due to the difference in cross-section existing between the first fluidic connecting element 20, in particular the injection orifice 22, and the reservoir 10.

Indeed, the injection orifice 22 advantageously has an opening diameter of very small size compared to the diameter of the reservoir 10, in the case of spherical species. However, this diameter is greater than or equal to the size of the particles leaving the reservoir 10, i.e. the platelets P and other products, Dk, resulting essentially from the fragmentation of the megakaryocytes. Preferably, the opening diameter is less than or equal to 1 mm, whereas as we have seen, the reservoir 10 has longitudinal dimensions in the millimeter to centimeter range, and in any event much greater than those of the orifice 22. It should be noted at this point that the shape of the injection orifice 22 is not limiting. What is important here is the size of said injection orifice 22.

Thus, an abrupt cross-sectional enlargement exists between the injection orifice 22 and the reservoir 10. This abrupt enlargement corresponds to a singularity and gives rise to a singular flow profile of the fluid F. As mentioned previously, the fluid F is accelerated out of the narrowing portion 24 through the injection orifice 22, and then subjects a pressure drop, i.e. a slowing, as it passes from the injection orifice 22 to the reservoir, due to this singularity. The smaller in front of 1 the ratio, R_{E}, of the diameter of the injection orifice 22 to the diameter or, more generally, the cross-sectional width of the reservoir 10, the greater the singularity and the greater the pressure drop, and vice versa as this ratio increases. An example of the system 1 according to the invention which has been implemented comprises a reservoir 10 with a diameter equal to 16 mm and an injection orifice 22 with a diameter equal to 0.8 mm (R_{E} is therefore equal to 0.05). If such a system 1 allows to provide improved platelet release efficiencies, ratios R_{E} of the opening diameter of the orifice 22 to the reservoir cross-sectional width of between 0.02 and 0.1 can also be envisaged for releasing platelets from a fluid F, always for an injection orifice 22 with an opening diameter of less than 1 mm. Advantageously, R_{E} is between 0.04 and 0.08 an opening diameter of less than 1 mm. More advantageously, R_{E} is between 0.04 and 0.06 an opening diameter of less than 1 mm. This being said, even more advantageously R_{E} is equal to 0.05. In fact, in the latter case, the efficiency of release of the platelets efficiency is better.

However, as will be explained below, although the fluid F is slowed down on entering the reservoir 10 and maintains a laminar flow regime in the strict sense of the term—the Reynold's number varying between 0, at the center of a vortex whose size is close to that of the reservoir 10, and 1500, at the level of the injection orifice 22—it is not immobile within said reservoir 10 because of the displacement of fluid generated by the pumping device 60. The reservoir 10 is thus only a place of passage for the fluid F, and is not a place of storage of said fluid F, i.e. a place in which the fluid is brought to stagnate when the system 1 is in operation.

In this regard, it should be noted that in addition to the reservoir 10 of release of the platelets, the system 1 may also comprises a source reservoir 40 for the storage of the fluid F connected to the first fluidic connecting element 20 to supply the reservoir. The fluid F may therefore be stored in the source reservoir 40 prior to its passage through the first element 20, but this is not mandatory. The reservoir 40 is therefore the most upstream element of the system 1 in the direction of the flow.

In addition, the system 1 may also comprise a reservoir 50 of reception of the fluid F. The reservoir 50 of reception of the fluid is connected to the second fluidic connecting element 30 for collecting said fluid to be sucked from the reservoir. In contrast to the source reservoir 40, the receiving reservoir 50 is the furthest downstream element of the system 1 in the direction of the flow. The arrangement of the latter will be explained later with respect to the second fluidic connecting element 30.

The reservoir 10 can thus be seen as central in that the system 1 may comprise upstream of it the source reservoir 40 and downstream of it the receiving reservoir 50 in the direction of the flow.

The second fluidic connecting element 30 is, in turn, a means for discharging the fluid F outside the reservoir 10 and thus forms an outlet channel for the fluid F. It is in fluidic communication, via one of its ends, with the receiving reservoir 50. In addition, it is attached at the level of the second opening 18 of the reservoir. It comprises an orifice 32 of discharging the fluid, a discharging portion 34 and a connecting portion 36 in that order in the direction of flow of the fluid F. It is arranged similarly to the first fluidic connecting element 10 relative to the reservoir 10, although oriented differently, and has a similar structure (e.g., size of the discharge orifice 32, relative to the reservoir 10 of platelet release).

That being said, the geometry of the flow is different at the reservoir 10/discharge orifice 32 interface, which is mainly due to the type of singularity. In contrast to the injection orifice 22/reservoir 10 interface, the singularity lies in an abrupt narrowing of the cross-section in the direction of the flow since the diameter (or circumference of the reservoir 10 if applicable) is much larger than the diameter of the discharge orifice 32. Given the dimensions of connecting reservoir 10 and the discharge orifice 32, such a singularity would further increase the pressure drop subjected by the fluid F as it passes from the reservoir 10 to the second fluidic connecting element 30, but this is mitigated by the fluid displacement generated by the pumping device 60, as will be described in more detail below. This singularity allows to increase the residence time of the fluid within the reservoir 10 since the abrupt narrowing of the cross-section acts as a barrier for the fluid F which is prevented from exiting the reservoir 10 immediately if not directly from the reservoir. The direct consequence of this is that, as the residence time of the fluid F is increased, the formation of the turbulence or vortex disturbances within the reservoir 10 is favored.

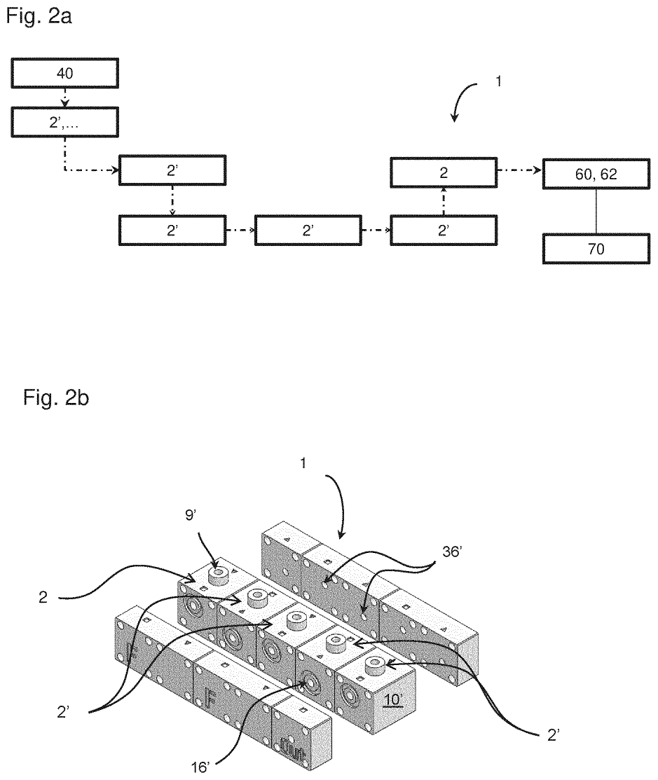

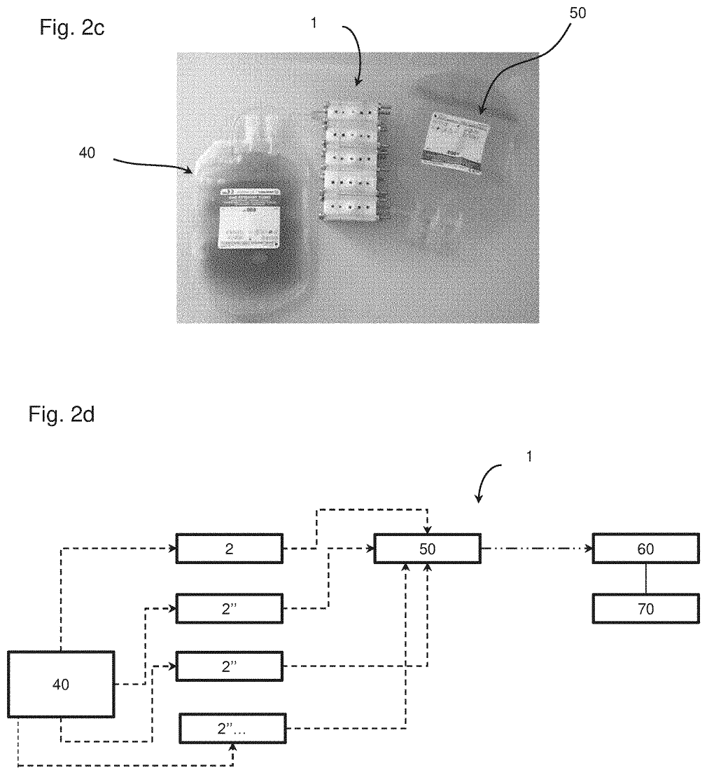

Furthermore, the residence time of the fluid F within the reservoir 10 can be further advantageously increased by offsetting the injection orifice 22 with respect to the discharge orifice 32. Indeed, the injection orifice 22 and the orifice 32 although being carried by two axes and thus forming a plane, this plane is oblique, i.e. it is neither horizontal nor vertical, as illustrated in the example of FIG. 1b. In this regard, as illustrated in FIG. 1c which represents a close-up view of the region R framed in FIG. 2b and as mentioned above, the first connecting element 20 comprises a longitudinal axis X1. The longitudinal axis X1 is a central axis of the first fluidic connecting element 20 passing through the injection orifice 22. The second fluidic connecting element 30, in turn, comprises a longitudinal axis X2 arranged in a similar manner as the longitudinal axis X1 with respect to said second fluidic connecting element 30. The longitudinal axes X1 and X2 may be intersecting or parallel. In order to increase the residence time of the fluid F and to increase the vortex disturbances, said longitudinal axes are advantageously separated by a non-zero distance, d. This configuration also allows a “bubble-free” filling of the reservoir 10. Indeed, the fluid F enters through the injection orifice 22, by suction, and exits through the discharge orifice once all the air contained in said reservoir 10 has been sucked out.

The discharge orifice 32 is thus separated by a non-zero distance, d, from the injection orifice 22 in a direction of axis Y, the axis Y being orthogonal to the longitudinal axes X1 and X2. Preferably, the distance, d, is between 3 and 4 mm. However, while said orifices 22, 32 may be separated by a distance d in the direction of axes Y, it is not excluded that they may be separated by a distance d′ in a direction of axis X in the same plane and/or in a plane XZ, the axis Z being orthogonal to the axis X in the direction of the cross-section (out of the plane of the figure). In this, the sectional view in FIG. 1c may be misleading since the first 16 and second 18 openings and the injection 22 and discharge orifice 32 respectively may be located in different cross-sectional planes (orthogonal to the axis Z).

If the geometry of the flow depends substantially on the singularities previously described and thus on the shape of the elements, it also depends on the flow speed. The shape of the elements of the device 2 and the flow speed thus act synergistically to achieve such a flow geometry and allow continuous release of platelets P from the megakaryocytic cells Mk with an improved efficiency compared to the known devices. We will come back to this in the following.

In this regard and as previously mentioned, the system 1 is provided with a pumping device 60 (illustrated in FIG. 1b). The pumping device 60 is in fluidic communication with the reservoir 10 of platelet release. It allows to move the fluid F contained in the reservoir 10. The displacement of the fluid may be due to a depression in the device 2, but it may also be due to a discharge of the fluid as will be seen in more detail later. What is important here is that the fluid F can flow with a higher velocity than it would have in the absence of the pumping device 60. The pumping device 60 is, for example, a vacuum pump or generally any device adapted to generate a depression or a fluid discharge in the device 2. For example, the pumping device 60 is a vane pump, but this is by no means limiting in the scope of the present invention.

Alternatively, the pumping device 60 may be located upstream of the first fluidic connecting element 20 in the direction of the flow, preferably in the source reservoir 40. In this regard, a pumping device 60 by fluid transfer or an overpressure in the reservoir 40 may be provided. In this configuration, the fluid F being discharged in the desired flow direction, it passes successively through the narrowing portion 24, the injection orifice 22, the reservoir 10 and the discharge orifice 32, adopting a flow geometry resulting from each of the singularities. However, the use of a fluid transfer pumping device 60 (gear pump, peristaltic pump, etc.) could be detrimental to the release of the platelets as it would likely degrade the megakaryocytic cells before the process has even begun. The use of this type of pumping device 60 is not mandatory since an ejector type device could also be considered. In general, any type of pumping device 60 may be used, with the possible exception of a fluid transfer pump.

The pumping device 60 may be in fluidic communication with the reservoir 10 by means of the second fluid connecting element 30. Preferably, it is located in the receiving reservoir 50. By being arranged in this way, the pumping device 60 is therefore located downstream of the second fluidic connecting element 30 in the direction of flow of the fluid F. This allows full advantage to be taken of the singularities of the device 2 and improves the process of forming vortex disturbances within the reservoir 10, in particular its cavity 14.

In this regard, let us return to the discharge portion 34 of the second connecting element 30. Advantageously, it may be flared from the discharge orifice 32 but this is not essential, to allow the formation of vortex disturbances within the reservoir 10. It should be noted that such a flaring of the discharge portion 34 would be due to the fact that its cross-section enlarges from the orifice 32 of discharging the fluid in the direction of the pumping device 60, i.e. in the direction of the flow. The flaring of the discharge portion 34 allows the pumping device 60, when located downstream of the second connecting element 30, to move the fluid more efficiently. This is simply because the pumping would not be as efficient if the second connecting element 30 had a cross-sectional area equal to that of the discharge orifice 32 along its entire length, its length being defined as the distance between the discharge orifice 32 and another end of said second element 30. Indeed, as mentioned above, the discharge orifice 32 has a diameter of at most 1 mm. The flaring is therefore of no interest other than to allow more efficient suction.

The system 1 further comprises a power supply module 62 for the pumping device 60. The power supply module 62 allows the pumping device 60 to act as a motor and to move the fluid F. In addition, the power supply module 62 also allows the power delivered by the pumping device 60 to be adjusted and any other useful parameters that the person skilled in the art will appreciate. Moreover, preferably, the power supply module 62 is configured so that the power delivered by the pumping device 60 allows the displacement speed of the fluid F in the system 1 to be adjusted to obtain a flow rate of fluid F of between 37 mL/min and 120 mL/min or even more. However, this is not the only parameter influencing the flow rate of fluid, as we will see in the following. Moreover, the system 1 may be equipped with a flow meter for controlling the flow rate of fluid in the reservoir 10 as a function of the power delivered by the power supply module 62. As an example, such a flow meter could be placed between the source reservoir 40 and the reservoir 10. Furthermore, the power supply module 62 need not be located in close proximity to the pumping device 60. It can be deported.

The system 1 further comprises a programming system 70 configured to control the power supply module 62 for the pumping device 60 to implement one or more platelet release sequences carried out so as to generate a continuous flow of the fluid F between said injection orifice 22 and said discharge orifice 32 and vortex disturbances within the reservoir 10 causing the fragmentation of the cytoplasmic extensions Ck of the megakaryocytic cells Mk. The programming system 70 comprises at least one or more processors adapted to execute a program to implement said platelet release sequences. As will be discussed below, the platelet release sequences may vary in their duration, their number, etc. That said, it should be noted that while the power supply module 62 may be controlled by means of such a programming system 70 for an industrial application, it may just as well be controlled manually by an operator.

With reference to FIGS. 2a to 2d, in a second and third embodiment of the invention, the system 1 comprises, in addition to the first device 2, a plurality of further devices 2′ or 2″. The other devices 2′ and 2″ are identical to the previously described device 2. The dotted arrowed lines indicate the direction of movement of the fluid F in the system.

In the second embodiment of the system 1 according to the invention illustrated in FIGS. 2a to 2c, the device 2 is connected in series with other devices 2′. The devices 2, 2′ are in fluidic communication with each other. Preferably, the number of further devices 2′ is between 1 and 4 so that the total number of devices 2, 2′ is between 1 and 5. In such a configuration, the system 1 allows very advantageously to multiply the number of singularities and thus to further improve the efficiency of release of the platelets. In effect, this multiplies the number of turbulences within the system 1 by the number of other devices 2′ so that each time the fluid F passes through another device 2′ the platelet release efficiency is increased.

An example of an embodiment of such an assembly is shown in FIG. 2b. It comprises five devices 2, 2′ mounted in series. The reservoirs 10′, the openings 16′ (the openings 18′ being opposite and not visible) are distinguished. The reservoirs 10′ have an outer shell of cubic shape, which does not prevent them from having a cavity 14 of any desired shape, here it is of spherical shape. In the illustrated embodiment, the reservoir 10′ is provided with a chimney 9′ to evacuate air bubbles. However, this is not mandatory. The connecting elements 20′, 30′, at least their respective portions 24′, 34′ and orifices 22′, 32′ are formed in assembling parts, located on both sides of the reservoirs 10′. The orifices 22′, 32′ are not aligned, i.e. they are offset from each other in the direction of flow of the fluid F, for each other device 2′.

In this regard, it may also be stated that the reservoir or the reservoirs 10, 10′, the aforementioned assembling elements and the connecting elements 20, 20′, 30, 30′ may be manufactured by any suitable manufacturing method known to the prior art. In the present case, the devices 2, 2′ were manufactured by 3D printing and assembled by screwing. In this regard, the devices 2, 2′ may comprise fastening means to allow their assembly. Preferably, they are made from polyether imide (PEI) resins or photopolymerizable resins (such as those used in the dental orthoses). Preferably, it is a material that complies with the pharmacopoeia of the country in which the system 1 according to the invention is to be used. The material can be certified by the ANSM (Agence Nationale de Sécurité du Médicament et des Produits de Santé), the EMA (European Medicines Agency) in France, the PhEU (European Pharmacopoeia), the PMDA (Pharmaceutical and Medical Devices Agency) in Japan or the FDA (Food and Drug Administration) and/or the USP (United States Pharmacopoeia) in the United States, etc. For example, for use in the United States, the device may preferably be made of biocompatible materials classified as USP VI, where VI refers to the USP class. The USP class test is one of the most common test methods for determining the biocompatibility of the materials. There are six classes, VI being the most rigorous. The class VI tests are intended to certify that there are no harmful reactions or long-term physical effects caused by chemicals released from plastics. Because of these specificities and the fact that the devices 2′ are closed, they are particularly well adapted to a treatment of the fluid specific to a medical use.

Such a configuration is also advantageous in that it improves the efficiency of the system while being optimally arranged. Indeed, instead of providing a pumping device 60 and a power supply 62 per device 2, 2′, these elements are shared by two, three or even more devices 2, 2′. Indeed, while the pumping device 60 is in fluidic communication with the first device 2 by means of the second fluidic connecting element 30 of said first device 2, as seen previously, the other devices 2′ are connected directly to the first connecting element 20, 20′ of the device 2, 2′ which precedes them. In other words, only the first device 2 is connected to the pumping device 60.

Consider, for example, the first other device 2′ directly connected to the first device 2. The second fluidic connecting element 30′ of said first other device 2′ is in fluidic communication with said first fluidic connecting element 20 of said first device 2. Preferably, this fluidic communication is direct. It is therefore in indirect fluidic communication with the pumping device 60 via the first device 2. Now consider the second other device 2′ directly connected to the first other device 2′. The second fluidic connecting element 30′ of said second other device 2′ is in fluidic communication with said first fluidic connecting element 20′ of said first other device 2′. This also applies to the other successive devices 2′.

Other types of assemblies, i.e. connection of the pumping device 60 to the devices 2, 2′ can be envisaged to connect the devices 2, 2′ in series while respecting the inventive concept of the invention.

It should be noted that, similarly to what has been seen for the first embodiment, the system 1 according to this second embodiment comprises in addition to the pumping device 60, the power supply module 62 for the pumping device 60 and the associated programming system 70. The geometry of the flow is not changed in such a system 1. In fact, the flow speed is preserved since instead of generating the depression or a discharge in the circuit of a single device 2, it propagates in all the devices 2′.

It may then be necessary to adapt the suction or discharge power, depending on the pumping device 60 used, and more precisely to increase it so that a sufficient displacement of the fluid is obtained for the most distant device 2′. However, if the suction or discharge power is too high, the efficiency of the platelet release can be adversely affected, since the efficiency is only optimal within a defined range of fluid flow rate and thus suction or discharge power. It is therefore appropriate to make such an adaptation according to this constraint if the pumping device 60 used does not allow a constant pressure to be maintained in the system 1. Most of the pumping devices currently available on the market generally allow to avoid this problem.

In a similar manner to what has been mentioned above for the pumping device 60, the power supply module 62 and the programming system 70, it is also possible to provide only a single source reservoir 40 and a single receiving reservoir 50 for the entire system 1, whether it comprises one or, if applicable, a plurality of devices 2. Such an embodiment is, for example, illustrated in FIG. 2c. In this example embodiment, the source reservoir 40 and the receiving reservoir 50 consist of flexible bags. The source reservoir 40 is in fluidic communication with the other device 2′ furthest upstream, i.e. furthest away from the first device 2 in the fluidic circuit. As for the receiving reservoir 50, similarly to the pumping device 60, it is in fluidic communication with the first device 2 by means of the second fluidic connecting element 30. It should be remembered that the pumping device 60 can advantageously be located in the receiving reservoir 50.

In a third embodiment of the invention shown in FIG. 2d, the system 1 comprises devices 2, 2″ connected in parallel, i.e. the first device 2 is connected in parallel with other devices 2″. In this configuration, the devices 2, 2″ are not connected to each other. In other words, they are independent of each other. Each device 2, 2″ thus operates independently of the others. The preferred number of device or devices 2″ is not limited. As the number of devices 2″ increases, the amount of fluid treated can be increased. For example, with a system 1 comprising three devices 2, 2″ connected in parallel, it is possible to treat about 3 liters of fluid F per hour, which gives the invention a considerable advantage over the devices known in the prior art. This configuration allows to adapt the number of devices 2, 2″ in operation according to the volume of fluid F to be treated. A larger volume source reservoir 40 can be provided but is not required. Alternatively, a means for continuously delivering the fluid to be treated could be provided.

In any case, similarly to the alternative embodiment of FIG. 2b, the pumping device 60, the power supply module 62 and the programming system 70 are mutualized for all the devices. However, in this case, the pumping device 60 can be put in fluidic communication with all the devices 2, 2″ by a set of connections. Other types of assemblies can be envisaged to mount the devices 2, 2″ in parallel while respecting the inventive concept of the invention.

Furthermore, the system 1 of the present invention can be adapted to integrate additional functionality, again in keeping with the inventive concept of the invention. For example, a pipe with a shape in Y could be provided at the inlet of the reservoir 10, which would allow two types of fluid F to circulate and mix within the reservoir 10, which, as we have seen, is configured to generate vortex disturbances.

The invention further relates to a method 5 for releasing platelets P from a fluid F comprising megakaryocytic cells Mk comprising cytoplasmic extensions Ck. The method 5 according to the invention allows, as will be discussed in detail in the following sections, to fragment megakaryocytes, Mk, and/or cytoplasmic extensions, Ck, in order to release the platelets. In this regard, the method 5 is implemented by means of a system 1 as previously described.

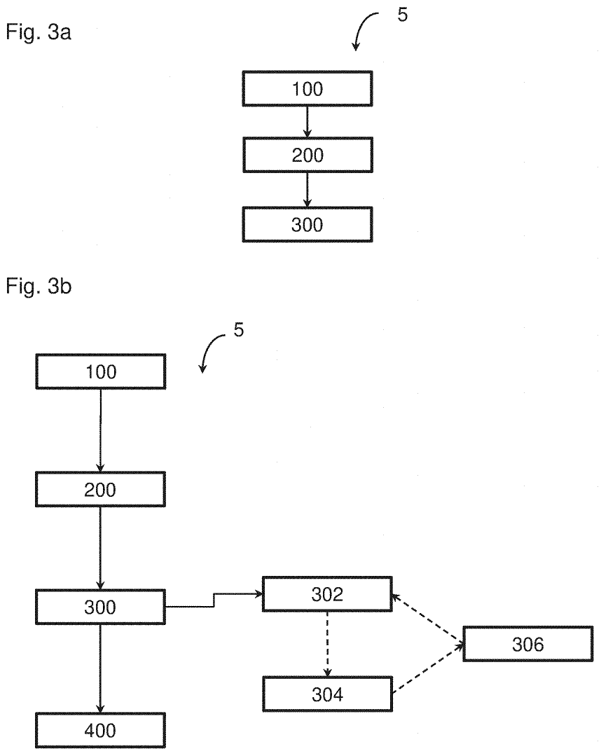

With reference to FIGS. 3a and 3b, during a first step 100 of the method 5, a fluid F comprising megakaryocytes Mk is provided. Preferably, the fluid F is stored in a source reservoir 40 provided for this purpose. The fluid F in question is, for example, a fluid taken from a patient, in particular from his bone marrow, or a fluid obtained by cell culture. In any case, this fluid F comprises megakaryocytes Mk and platelets may be released from it. However, it should be noted that whatever the fluid F, the megakaryocytes Mk are suspended in said fluid F and that, depending on the viscosity of the fluid, the megakaryocytes will be distributed differently.

Considering the industrial quantities of fluid F that can be treated and incidentally of platelets that can be released, a preliminary cell culture step can be considered in order to obtain the desired quantity of fluid F and precursors (i.e. megakaryocytes). The purpose of this cell culture step is to allow the maturation of the megakaryocytes Mk and their multiplication, i.e. their proliferation. The document FR 3 039 166 describes the cell culture process in more detail.

For example, the megakaryocytes Mk used in the Fluid F are megakaryocytes derived from immortalized or non-immortalized CD34+ progenitors. The pre-cultivation step of such megakaryocytes Mk may comprise two phases lasting a total of 11-17 days. During a first phase lasting between 5 and 9 days, the megakaryocytes Mk are cultured with a mixture comprising, for example, the following agents: serum-free culture medium, a cytokine cocktail containing TPO, IL-6 and IL-9 and the AhR antagonist and the LDL (Low density lipoprotein intended to stimulate the proliferation of the cells CD34+ and to engage them in the pathway of the megakaryocytopoiesis). The AhR favorably stimulates the maturation of the megakaryocytes Mk. In a second phase of this culture step, which lasts approximately 6-8 days, the resulting cells are cultured with a mixture of SR1 and thrombopoietin, TPO, and optionally other agents such as listed above with reference to the first phase. It should be noted that the cells obtained comprise megakaryocytes but not exclusively. In addition, it should be noted that not all cells give rise to megakaryocytes. These phases can last more or less time depending on the number of cells desired, the agents used, etc.

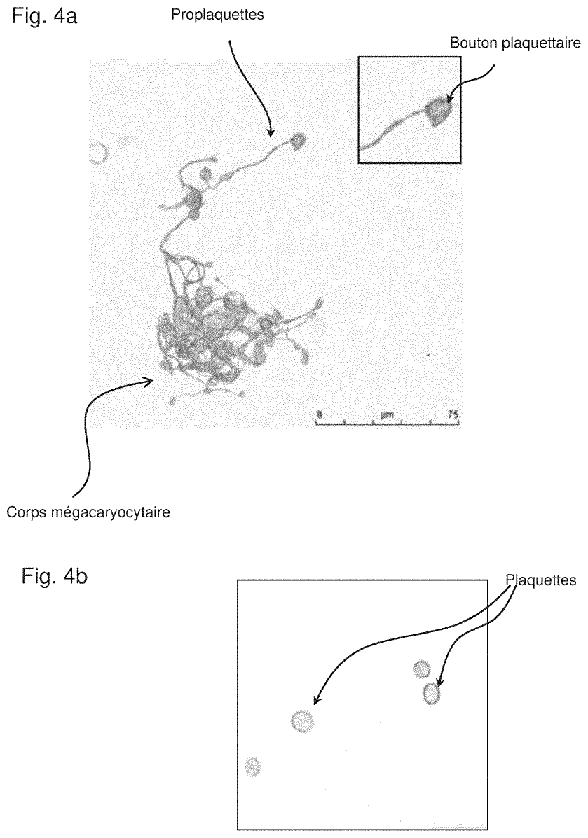

This preliminary step will not be further described as it is not the subject of the present invention and does not form part of it, the invention being limited to propose a method for the release of the platelets from the fluid F and not a method comprising any culture step. In any case, at the end of this cell culture phase, proplatelet megakaryocytes are obtained, i.e. megakaryocytes provided with proplatelets. They are simply referred to as megakaryocytes Mk in this present invention for simplicity. Such cells are shown in FIG. 4a. They comprise a megakaryocytic body with proplatelets forming extensions from the megakaryocytic body. The cytoplasmic extensions are supported by a network of microtubules and terminate in a platelet button that prefigures the future platelet. The fluid F provided in the step 100 comprises such proplatelet megakaryocytes.

Referring again to FIGS. 3a and 3b, the method 5 comprises subsequent to the step 100, a step 200 in which the pumping device 60 is electrically powered so as to generate a depression or a discharge at the outlet of the second fluidic connecting element 30.

During the step 200, the power of the power supply module 62 for the pumping device 60 may be set so as to adjust the suction or discharge power. Specifically, the power should be adjusted so that the resulting flow rate of fluid F is between 37 mL/min and 120 mL/min or more. It should be noted that depending on the material of which the device of the system 1 according to the invention is made, the fluid flow rate can be significantly increased. This is the optimum flow rate range to achieve the highest platelet release efficiencies with the method according to the invention. The power to be applied is inherently dependent on the material used for the pumping device 60.

During a step 300, the programming system 70 is controlled to initiate one or more platelet release sequences as previously defined. Advantageously, the programming system 70 can control the power supply module 62. It also allows to control over the times and the number of platelet P release sequences. The platelet release sequences can last as long as the user wishes in order to treat the desired volume of fluid.

When the programming system 70 as described in the previous section is operated with the pumping device 62 powered, a depression or, depending on the configuration, a discharge is generated in the device or the devices 2, 2′, 2″. “depression” means that the pressure in the device or the devices 2, 2′, 2″ is lower than the atmospheric pressure, i.e. that it/they is/are subjected to a relative vacuum. The “discharge” corresponds to the fact of moving the fluid F by a discharge pump, for example an ejector, or a compressor. As a result of this depression or this discharge, a continuous flow of fluid F, i.e. a displacement takes place in the system 1 from the source reservoir 40 to the receiving reservoir 50. It should be noted that, preferably, the suction or discharge power of the pumping device 60 is kept constant during the method 5 so that the depression, where appropriate the discharge speed, and the flow rate of fluid F are also kept substantially constant. However, this does not prevent the power from being varied during the implementation of the method within limits that allow the optimum platelet release efficiency to be obtained.

As soon as the depression or the discharge is generated in the system or the systems 2, 2′, 2″, the fluid goes through different phases i.e. 302, 304 and 306, which occur continuously as long as the depression or the discharge is maintained, without further action on the programming system 70. These phases are the direct consequence of the fluid displacement in the system or the systems 2, 2′, 2″. A phase 302 corresponds to the phase before the fluid F enters the reservoir 10, a phase 304 corresponds to the phase when the fluid F is in the reservoir 10 and a phase 306 corresponds to the phase after the fluid F is sucked out of the reservoir 10. In other words, the phases 302, 304 and 306 occur in practice simultaneously since when a part of the fluid F is being treated, at the same time another portion of the fluid F is being treated in the reservoir 10 and another part is about to be treated upstream of the reservoir 10, i.e. in the first connecting element 20 or in the source reservoir 40, along the fluidic circuit.

During the phase 302, the fluid F is sucked through the first fluidic connecting element 20. As the pressure within the device is maintained constant, the speed of the fluid F increases as it passes through the narrowing portion 24, due to the very fact of the flaring of said narrowing portion 24. The fluid F is then discharged into the reservoir 10 via the injection orifice 22. The singularity due to the abrupt cross-sectional enlargement present at the injection orifice 22/reservoir 10 interface causes a slowing down of the fluid F when it enters the reservoir 10.

During the phase 304, the fluid F flows through the reservoir 10 being subjected to the depression or the discharge generated by the pumping device 60 within the limits of the cavity 14. Indeed, although the fluid has been slowed down by the singularity existing at the orifice 22/reservoir 10 interface, it remains in motion because of this depression or discharge. The fluid does not flow directly out of the reservoir, which allows the circulation of said fluid in the form of vortex disturbances. Indeed, the system 1 is configured so that the residence time of the fluid F in the reservoir 10 is sufficient to generate disturbances. As previously discussed, a sufficient residence time is achieved due to the singularity of the large size of the reservoir 10 relative to the discharge orifice 32. The residence time can also be increased, but to a lesser extent, by advantageously misaligning the injection orifice 22 with respect to the discharge orifice 32 in the path of the fluid F as explained below.

Under these conditions, vortex disturbances of approximately the same size as megakaryocytes are generated. They allow to fragment the megakaryocytes Mk and their cytoplasmic extensions Ck (proplatelets), resulting in the release of the platelets P. At the same time, other products Dk resulting from the fragmentation of megakaryocytes are produced. These products are bits of proplatelets, intact bodies of megakaryocyte Mk or pieces of cytoplasm. Since the fluid F is treated continuously over time for each platelet release sequence, the phenomena described above are reproduced continuously and the platelets are released continuously. In sum, while the fluid F entering the reservoir 10 comprises megakaryocytic cells Mk comprising cytoplasmic extensions, the fluid F exiting the reservoir 10 comprises essentially platelets P and the other products, Dk. In this regard, the use of a system 1 comprising devices 2, 2′ in series, as illustrated in FIGS. 2a to 2c, advantageously allows to reduce the proportions of megakaryocytes still intact following the method 5. It should be remembered that each time the fluid F circulates in a device 2′, it undergoes disturbances, hence the higher efficiencies of a system 1 in series compared with a system 1 comprising a single device 2.

During the phase 306, the fluid F, loaded with platelets P and other products Dk resulting from the fragmentation of the megakaryocytes is sucked through the discharge orifice 32 and more generally through the second fluidic connecting element 30. It then reaches the receiving reservoir 50, if any, and the pumping device 60.

During a step 400, the sequence or the sequences are stopped by shutting down the power supply module 62 using the programming system 70.

These phases occur identically in any other devices 2′ and/or 2″ present in the system 1 according to the invention.

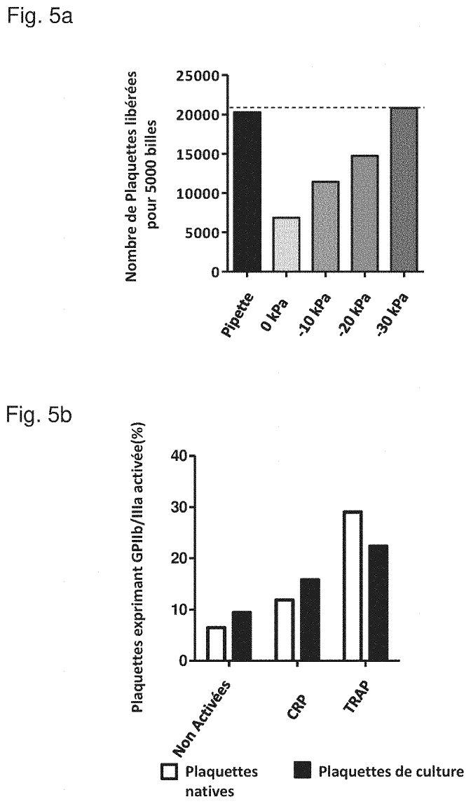

In order to release the platelets, the flow rate of fluid should advantageously be between 37 mL/min and 120 mL/min or more. These flow rate values are average values representing the average of six measurements. FIG. 5a illustrates the evolution of the number of platelets for a number of calibration tubes (BD Trucoun® Tubes) or counting tubes equal to 5000 as a function of the relative vacuum generated by implementing the method according to the invention (grey bars) and by means of a pipette (black bar). The relative vacuum is in particular expressed in kilo Pascal (kPa). When no depression or discharge is generated in the device or the devices 2, 2′, 2″, the platelet release efficiency remains very low (very light grey). With a relative vacuum equal to −10 kPa, the minimum value of 37 mL/min mentioned above is obtained. With such a relative vacuum, the platelet release efficiency increases and the number of released platelets exceeds 10,000 per 5,000 tubes. This efficiency of release of the platelets is still relatively low. As the relative vacuum increases, the efficiency of release of the platelets increases. When the relative vacuum is equal to −30 kPa, the previously mentioned value of 120 mL/min is obtained. The number of platelets released is more than 20,000 per 5,000 megakaryocyte tubes. As shown in FIG. 5a, the resulting platelet release efficiency is close to and even higher than that obtained using a conventional manual pipette. This illustrates the importance of the depression or the fluid discharge generated in the system 1 in order to achieve a satisfactory release efficiency. If in the example embodiment shown here the relative vacuum is −30 kPa, it can be increased beyond −30 kPa, i.e. up to 50 kPa. The obtained fluid flow rate is well above 120 mL/min.

This is due to the characteristics of the device or the devices 2,2′, 2″ used in the present example embodiment. Indeed, to adjust the flow rate, a compromise must be found between the suction or discharge power of the pumping device 60, the sizes of the injection orifices 22 and of the discharge orifices 32, but also the condition of the surface of the elements of the device or the devices 2, 2′, 2″ through which the fluid F actually transits, this within the limits of the invention. Concerning this last aspect, the more the surface condition (micro-roughness, roughness, etc.) will have the effect of exerting constraints on the fluid F and the more it will be slowed down and vice versa. In order to achieve an even higher platelet release efficiency, the surface condition can be adjusted, for example. This can be done by modifying the material used to manufacture the elements of the device or the devices 2, 2′, 2″ through which the fluid F flows, by using other manufacturing methods allowing a control of the structuring on a micrometric scale but also by infusing a compound into the chamber such as plasma or albumin.

FIG. 5b shows an analysis of the functionality of the platelets obtained at a pressure of −30 kPa for native platelets (hollow bars) and cultured platelets (solid bars). With this relative vacuum, approximately 8% of native platelets and 10% of non-activated cultured platelets express the Glycoprotein GPIIb/IIIa. About 12% of the native platelets and 16% of the cultured platelets activated with a agonist of the CRP type (Collagen Related Peptide) express the glycoprotein GPIIb/IIIa. Finally, about 30% of the native platelets and 22% of the cultured platelets activated with a agonist of the TRAP type (Trombin Receptor-Activating Peptides) express the glycoprotein GPIIb/IIIa. The platelets obtained by the method according to the invention are therefore well activated and consequently functional.

1. A system for releasing platelets from a fluid (F) comprising megakaryocytic cells (Mk) comprising cytoplasmic extensions (Ck), said system comprising:
a device comprising:
a platelet release reservoir comprising a first opening and a second opening,
a first fluidic connecting element attached at the level of said first opening and adapted to inject said fluid (F) into said reservoir, said first connecting element comprising an orifice for injecting the fluid into the platelet release reservoir and a portion narrowing towards said orifice, there being an abrupt widening of cross-section between the injection orifice and the reservoir,
a second fluidic connecting element attached at the level of said second opening, said second connecting element comprising an orifice for discharging the fluid,
a device for pumping the fluid (F) in fluidic communication with the reservoir by the second fluidic connecting element or the first fluidic connecting element,
a power supply module for the pumping device,
a programming system configured to control the power supply module for the pumping device, to implement one or more platelet release sequences designed to generate a continuous flow of the fluid (F) between said injection orifice and said discharge orifice and vortex disturbances within the reservoir causing the fragmentation of the cytoplasmic extensions (Ck) of the megakaryocytic cells (Mk).

2. The system according to claim 1, wherein the narrowing portion is conical.

3. The system according to claim 1, wherein the first connecting element comprises a longitudinal axis (X1) and the second connecting element comprises a longitudinal axis (X2), said longitudinal axes (X1, X2) being either intersecting or parallel and then separated by a non-zero distance, d.

4. The system according to claim 1, wherein the injection orifice has an opening diameter of less than 1 mm, a ratio of the opening diameter of the injection orifice by a sectional width of the reservoir is between 0.02 and 0.1.

5. The system according to claim 4, wherein the ratio of the opening diameter of the injection orifice to the cross-sectional width of the reservoir is 0.05.

6. The system according to claim 1, wherein the discharge orifice has an opening diameter of less than 1 mm, a ratio of the opening diameter of the discharge orifice to a sectional width of the reservoir is between 0.02 and 0.1.

7. The system according to claim 6, wherein the ratio of the opening diameter of the discharge orifice to the cross-sectional width of the reservoir is 0.05.

8. The system according to claim 1, wherein the reservoir has a spherical shape.

9. The system according to claim 1, comprising:
a source reservoir for the storage of the fluid (F), connected to the first fluidic connecting element for supplying the reservoir and
a reservoir for receiving the fluid (F) connected to the second fluidic connecting element to collect said fluid intended to be sucked from the reservoir.

10. The system according to claim 9, wherein the pumping device is located in the receiving reservoir.

11. The system according to claim 1, wherein said second connecting element further comprises a portion flared from said orifice for discharging the fluid towards the pumping device.

12. The system according to claim 1, comprising at least one other device comprising:
a platelet release reservoir comprising a first opening and a second opening,
a first fluidic connecting element attached at the level of said first opening and adapted to inject said fluid (F) inside said reservoir, said first connecting element comprising an orifice for injecting the fluid and a first narrowing portion so as to be able to accelerate the fluid (F), said first narrowing portion opening onto said injection orifice,
a second fluidic connecting element (30″) attached at the level of said second opening, said second connecting element comprising an orifice for discharging the fluid, said other device being arranged parallel to the first device and connected to the pumping device by the second element of said other device.

13. The system according to claim 1 comprising at least one other device comprising:
a platelet release reservoir comprising a first opening and a second opening,
a first fluidic connecting element attached at the level of said first opening and adapted to inject said fluid (F) inside said reservoir, said first connecting element comprising an orifice for injecting the fluid and a first narrowing portion so as to be able to accelerate the fluid (F), said first narrowing portion opening out on said injection orifice,
a second fluidic connecting element attached at the level of said second opening, said second connecting element comprising an orifice for discharging the fluid, said other device being arranged in series with the first device, said second connecting element of said other device being in fluidic communication with said first fluidic connecting element of said first device.

14. A method for releasing platelets (P) from a fluid (F) comprising in particular megakaryocytic cells (Mk) comprising cytoplasmic extensions (Ck), said method comprising the following steps, implemented by the system according to claim 1:
providing a fluid (F) comprising megakaryocytic cells (Mk) suspended in said fluid (F), said megakaryocytic cells (Mk) comprising cytoplasmic extensions (Ck),
electrically powering the pumping device, and
controlling the programming system to initiate one or more platelet release sequences, the or each platelet release sequence being carried out so as to generate a continuous flow of the fluid (F) between said injection orifice and said discharge orifice and vortex disturbances within the reservoir causing the fragmentation of the cytoplasmic extensions (Ck) of the megakaryocytic cells (Mk).

15. The method according to claim 14, wherein during the step of electrically powering the pumping device a relative vacuum of between −10 kPa and −50 kPa is generated in the device.
